(19) United States
(12) Patent Application Publication
(10) Pub. No.: US 2016/0029982 A1
(43) Pub. Date: Feb. 4, 2016

(54) MEDICAL IMAGE DIAGNOSTIC APPARATUS, X-RAY CT APPARATUS, AND DETECTOR

(71) Applicants: Kabushiki Kaisha Toshiba, Minato-ku (JP); Toshiba Medical Systems Corporation, Otawara-shi (JP)

(72) Inventors: Emi TAMURA, Nasushiobara (JP); Yasuo SAITO, Nasushiobara (JP); Hiroaki MIYAZAKI, Otawara (JP)

(73) Assignees: Kabushiki Kaisha Toshiba, Minato-ku (JP); Toshiba Medical Systems Corporation, Otawara-shi (JP)

(21) Appl. No.: 14/748,457

(22) Filed: Jun. 24, 2015

(30) Foreign Application Priority Data
Jul. 30, 2014 (JP) 2014-155541

(51) Int. Cl. A61B 6/00; G01T 1/29; A61B 6/03

(52) U.S. Cl. CPC A61B 6/4241; A61B 6/035; G01T 1/2921

(57) ABSTRACT

A medical image diagnostic apparatus according to an embodiment includes detecting elements and processing circuitry. Each of the detecting elements contains, in an effective area thereof, a plurality of cells each configured to output an electrical signal when at least one photon has become incident thereto. Processing circuitry generates an image on the basis of a signal obtained by adding together the electrical signals output by the plurality of cells. The medical image diagnostic apparatus according to the embodiment includes a plurality of arrays in each of which two or more of the detecting elements containing an equal quantity of cells in the effective areas thereof are arranged, while the plurality of arrays are arranged in such a manner that the distances between the centers of the effective areas are constant.

### CROSS-REFERENCE TO RELATED APPLICATION

This application is based upon and claims the benefit of priority from Japanese Patent Application No. 2014-155541, filed on Jul. 30, 2014 the entire contents of all of which are incorporated herein by reference.

### FIELD

Embodiments described herein relate generally to a medical image diagnostic apparatus, an X-ray Computed Tomography (CT) apparatus, and a detector.

### BACKGROUND

Nuclear medical imaging apparatuses such as Positron Emission Tomography (PET) apparatuses and Single Photon Emission Computed Tomography (SPECT) apparatuses each include a photon-counting-type detector configured to detect radiation. In addition, X-ray Computed Tomography (CT) apparatuses including a photon-counting-type detector have been developed in recent years. Examples of the photon-counting-type detectors include a detector that has a plurality of silicon photomultiplier (SiPM) arrays each of which includes a plurality of silicon photomultipliers (SiPMs). Further, photon-counting-type detectors can be used not only for medical purposes, but also for industrial purposes.

Generally speaking, it is desirable that detectors have a larger area. Manufacturing steps of a detector include a step of arranging substrates of silicon photomultiplier arrays that are cut out from a silicon wafer to be positioned next to one another. By performing this step, it is possible to manufacture a detector having a large area while improving the yield of silicon wafers and reducing manufacturing costs.

In this situation, during the process of arranging the substrates to be positioned next to one another, the substrates may mechanically or electrically be damaged, if any of the substrates come in contact with each other. To avoid the damages, it is required that the substrates be positioned next to one another at constant intervals. Further, to enhance the image quality, it is required that the distances between the centers of any two silicon photomultipliers positioned adjacent to each other are constant in the direction of alignment.

To meet both of the two requirements, such silicon photomultipliers that are positioned at the ends or the four corners of each of the silicon photomultiplier arrays need to have a smaller area. The number of cells contained in the smaller silicon photomultipliers is smaller than the number of cells contained in the other silicon photomultipliers. Responsive characteristics of the silicon photomultipliers with respect to X-rays are dependent on the number of cells contained therein. For this reason, there are some situations where the image quality may be degraded by the difference in the responsive characteristics between the silicon photomultipliers having the smaller area and the other silicon photomultipliers.

### BRIEF DESCRIPTION OF THE DRAWINGS

FIG. 1 is a diagram of an exemplary configuration of a medical image diagnostic apparatus according to an embodiment;

FIG. 2 is a drawing of an example of a configuration and a positional arrangement of silicon photomultiplier arrays included in a conventional medical image diagnostic apparatus;

FIG. 3 is a drawing of another example of a configuration and a positional arrangement of silicon photomultiplier arrays included in a conventional medical image diagnostic apparatus;

FIG. 4 is a diagram of an example of a positional arrangement of cells;

FIG. 5 is a chart illustrating a relationship between the number of visible light photons detected by a silicon photomultiplier and X-ray energy and a relationship between the true number of visible light photons that are incident to the silicon photomultiplier and X-ray energy;

FIG. 6 is a drawing of an example of a configuration and a positional arrangement of silicon photomultiplier arrays included in the medical image diagnostic apparatus according to the embodiment;

FIG. 7 is a drawing of another example of a configuration and a positional arrangement of silicon photomultiplier arrays included in the medical image diagnostic apparatus according to the embodiment;

FIG. 8 is a diagram of an exemplary configuration of a medical image diagnostic apparatus according to a modified example; and

FIG. 9 is a flowchart of an example of a process performed by the medical image diagnostic apparatus according to the modified example.

### DETAILED DESCRIPTION

A medical image diagnostic apparatus according to an embodiment includes detecting elements and processing circuitry. Each of the detecting elements contains, in an effective area thereof, a plurality of cells each configured to output an electrical signal when at least one photon has become incident thereto. Processing circuitry generates an image on the basis of a signal obtained by adding together the electrical signals output by the plurality of cells. The medical image diagnostic apparatus according to the embodiment includes a plurality of arrays in each of which two or more of the detecting elements containing an equal number of cells in the effective areas thereof are arranged, while the plurality of arrays are arranged in such a manner that the distances between the centers of the effective areas are constant.

A medical image diagnostic apparatus, an X-ray CT apparatus, and a detector according to the embodiment will be explained below, with reference to the accompanying drawings.

### Exemplary Embodiments

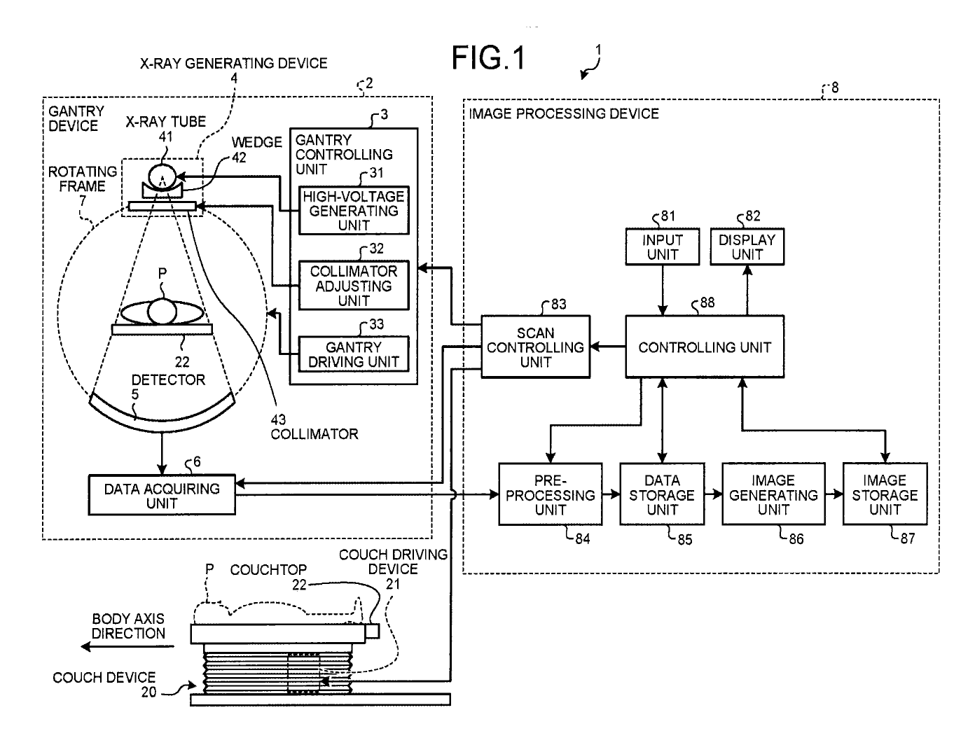

First, a configuration of a medical image diagnostic apparatus 1 according to the embodiment will be explained, with reference to FIG. 1. FIG. 1 is a diagram of an exemplary configuration of the medical image diagnostic apparatus 1 according to the embodiment. The medical image diagnostic apparatus 1 is a photon-counting-type X-ray CT apparatus. As illustrated in FIG. 1, the medical image diagnostic apparatus 1 includes a gantry device 2, a couch device 20, and an image processing device 8. Possible configurations of the medical image diagnostic apparatus 1 are not limited to the configuration described below.

The gantry device 2 acquires projection data (explained later) by irradiating X-rays with a subject P. The gantry device 2 includes a gantry controlling unit 3, an X-ray generating device 4, a detector 5, a data acquiring unit 6, and a rotating frame 7.

The gantry controlling unit 3 is configured, under control of a scan controlling unit 83 (explained later), to control operations of the X-ray generating device 4 and the rotating frame 7. The gantry controlling unit 3 includes a high-voltage generating unit 31, a collimator adjusting unit 32, and a gantry driving unit 33. The high-voltage generating unit 31 supplies an X-ray tube voltage to an X-ray tube 41 (explained later). The collimator adjusting unit 32 adjusts the radiating range of the X-rays radiated from the X-ray generating device 4 and irradiated the subject P, by adjusting the degree of aperture and the position of a collimator 43. For example, by adjusting the degree of aperture of the collimator 43, the collimator adjusting unit 32 adjusts the radiating range of the X-rays, i.e., the fan angle and the cone angle of the X-rays. The gantry driving unit 33 causes the X-ray generating device 4 and the detector 5 to rotate a circular trajectory centered on the subject P, by driving the rotating frame 7 to rotate.

The X-ray generating device 4 generates the X-rays to be irradiated the subject P. The X-ray generating device 4 includes the X-ray tube 41, a wedge 42, and the collimator 43. The X-ray tube 41 generates the X-rays in a beam form to be irradiated the subject F, by using the X-ray tube voltage supplied by the high-voltage generating unit 31. The X-ray tube 41 is a vacuum tube configured to generate the X-rays in the beam form spreading in a cone- or pyramid-shape, along the body axis direction of the subject P. The X-rays in the beam form may be referred to as “cone beams”. The X-ray tube 41 irradiates the subject P with the cone beams, in conjunction with the rotation of the rotating frame 7. The wedge 42 is an X-ray filter used for adjusting the X-ray dose of the X-rays radiated from the X-ray tube 41. The collimator 43 is a slit used for narrowing the radiating range of the X-rays of which the X-ray dose is adjusted by the wedge 42, under the control of the collimator adjusting unit 32.

The detector 5 includes detecting elements each of which contains, in an effective area thereof, a plurality of cells each of which outputs an electrical signal when at least one photon has become incident thereto. The detector 5 includes a plurality of arrays in each of which two or more of the detecting elements containing an equal number of cells in the effective areas thereof are arranged. The plurality of arrays are arranged in such a manner that the distances between the centers of the effective areas are constant. For example, the cells are Avalanche Photodiodes (APDs). For example, the detecting elements are silicon photomultipliers (SiPMs). In the following explanation, the silicon photomultipliers will be referred to as SiPMs. Further, the detector 5 includes scintillators and detecting circuits. One scintillator and one detecting circuit are installed in correspondence with each of the SiPMs. The photons of the X-rays that have become incident to the detector 5 are converted into visible light photons by the scintillators. The higher the energy of the X-rays is, the larger numbers of visible light photons are generated by the scintillators. The visible light photons are converted by the cells into predetermined electrical signals in a pulse form. The electrical signals are added together, and the result of the addition is transmitted to the data acquiring unit 6 for each of the silicon photomultiplier arrays, for example. Specifically, the electrical signals are added together for each of the SiPMs, and the result of the addition is transmitted to the data acquiring unit 6 for each of the silicon photomultiplier arrays. The detector 5 including the scintillators and the photodiodes is called an indirect-conversion-type detector. Further, the silicon photomultiplier arrays may simply be referred to as arrays. In the following explanation, the silicon photomultiplier arrays will be referred to as SiPM arrays. Details of a configuration and operations of the detector 5 will be described later.

The data acquiring unit 6 acquires count data, on the basis of the pulse-form signals obtained by adding together the predetermined pulse-form electrical signals output by the cells for each of the SiPMs. The count data is data in which a position of the X-ray tube 41, a position of the SiPM, and a count value of the incident visible light photons are associated with one another for each of a plurality of energy bins that are set in an X-ray energy distribution of the X-rays radiated by the X-ray tube 41. In this situation, the position of the X-ray tube 41 will be referred to as a “view”. On the basis of the waveform of a signal obtained by adding together the electrical signals output by the cells for each of the SiPMs, the data acquiring unit 6 is able to calculate the energy of the photons that have caused the signal to be output. For this reason, the data acquiring unit 6 is able to acquire the count data for each of the energy bins. The data acquiring unit 6 is able to calculate and acquire the count value of the visible light photons incident to each of the SiPMs, on the basis of the height of the peak of the pulse-form signal obtained by adding together, for each of the SiPMs, the predetermined pulse-form electrical signals output by the cells.

The data acquiring unit 6 generates the projection data on the basis of the acquired count values of the visible light photons. The count data is acquired for each of the energy bins that are set in the X-ray energy distribution of the X-rays radiated by the X-ray tube 41. Accordingly, the projection data are generated by the equal number of energy bins. The count values of the visible light photon are expressed as brightness values of the pixels corresponding to each of mutually-different views of the projection data. Alternatively, the count values of the visible light photons may be expressed as values per unit time. The data acquiring unit 6 transmits the generated projection data to a preprocessing unit 84. The data acquiring unit 6 may be called a Data Acquisition System (DAS).

The rotating frame 7 is an annular frame that supports the X-ray generating device 4 and the detector 5 so as to oppose each other while the subject P is interposed therebetween. The rotating frame 7 is driven by the gantry driving unit 33 and rotates on a circular trajectory centered on the subject P at a high speed. The rotating frame 7 and the gantry driving unit 33 may collectively be referred to as a rotating unit. The rotating unit rotates the X-ray tube 41 and the detector 5.

The couch device 20 includes a couch driving device 21 and a couchtop 22. The couch driving device 21 is configured, under the control of the scan controlling unit 83 (explained later), to move the subject P on the inside of the rotating frame 7, by moving the couchtop 22 on which the subject P is placed in a body axis direction. For example, the gantry device 2 performs a helical scan to helically scan the subject P, by causing the rotating frame 7 to rotate while moving the couchtop 22. Alternatively, the gantry device 2 performs a conventional scan to scan the subject P by moving the couchtop 22 and subsequently causing the rotating frame 7 to rotate while the position of the subject P is fixed. Alternatively, the gantry device 2 implements a step-and-shoot method by which the conventional scan is performed in a plurality of scanning areas by moving the position of the couchtop 22 at predetermined intervals.

The image processing device 8 receives operations performed by a user on the medical image diagnostic apparatus 1. Further, the image processing device 8 performs various types of image processing such as a reconstruction of the projection data acquired by the gantry device 2. The image processing device 8 includes an input unit 81, a display unit 82, the scan controlling unit 83, the preprocessing unit 84, a data storage unit 85, an image generating unit 86, an image storage unit 87, and a controlling unit 88.

The input unit 81 is a mouse, a keyboard, and/or the like used by the user of the medical image diagnostic apparatus 1 for inputting various types of instructions and various types of settings. The input unit 81 transfers information about the instructions and the settings received from the user to the controlling unit 88. The display unit 82 is a monitor referenced by the user. The display unit 82 displays results of various types of image processing, a Graphical User Interface (GUI) used for receiving the various types of settings from the user via the input unit 81, and the like.

The scan controlling unit 83 is configured, under the control of the controlling unit 88, to control operations of the gantry controlling unit 3, the data acquiring unit 6, and the couch driving device 21. Specifically, by controlling the gantry controlling unit 3, the scan controlling unit 83 causes the rotating frame 7 to rotate, causes the X-rays to be radiated from the X-ray tube 41, and adjusts the degree of aperture and the position of the collimator 43, when a photon counting CT imaging process is performed. Further, under the control of the controlling unit 88, the scan controlling unit 83 controls the data acquiring unit 6. Further, under the control of the controlling unit 88, the scan controlling unit 83 moves the couchtop 22 by controlling the couch driving device 21, when a photon counting CT imaging process is performed.

The preprocessing unit 84 performs a correcting process such as a logarithmic transformation, an offset correction, a sensitivity correction, a beam hardening correction, a scattered ray correction, or the like, on the projection data generated by the data acquiring unit 6. The preprocessing unit 84 stores the projection data on which the correcting process has been performed, into the data storage unit 85. The projection data on which the correcting process has been performed by the preprocessing unit 84 may be referred to as “raw data”.

The data storage unit 85 stores therein the raw data, i.e., the projection data on which the correcting process has been performed by the preprocessing unit 84. The image generating unit 86 generates an image on the basis of the signals obtained by adding together the electrical signals output by the plurality of cells. Specifically, for example, the image generating unit 86 generates the image on the basis of the signals that result from the addition for each of the SiPMs and are output in correspondence with the SiPM arrays. The image generating unit 86 generates a reconstructed image by reconstructing the projection data stored in the data storage unit 85. The reconstruction method may be selected from various methods including methods implemented by performing a back projection process, for example. The back projection process may be performed by using a Filtered Back Projection (FBF) method, for example. The image generating unit 86 may perform the reconstructing process by implementing a successive approximation method, for example. Further, the image generating unit 86 is also capable of generating a reconstructed image for each of substances distinguished by a substance distinguishing process. The image storage unit 87 stores therein the reconstructed image generated by the image generating unit 86.

The controlling unit 88 controls the medical image diagnostic apparatus 1 by controlling operations of the gantry device 2, the couch device 20, and the image processing device 8. The controlling unit 88 controls the scan controlling unit 83 so as to perform a scan and acquires the projection data from the gantry device 2. Further, the controlling unit 88 controls the preprocessing unit 84 so as to apply the abovementioned correcting process to the projection data. Further, the controlling unit 88 controls the display unit 82 so as to display the projection data stored in the data storage unit 85 and image data stored in the image storage unit 87.

The data storage unit 85 and the image storage unit 87 described above may be realized by using, for example, a semiconductor memory element, a hard disk, or an optical disk. The semiconductor memory element may be, for example, a Random Access Memory (RAM) or a flash memory. The scan controlling unit 83, the preprocessing unit 84, the image generating unit 86, and the controlling unit 88 described above may be realized by using an integrated circuit or an electronic circuit. The integrated circuit may be, for example, an Application Specific Integrated Circuit (ASIC) or a Field Programmable Gate Array (FPGA). The electronic circuit may be, for example, a Central Processing Unit (CPU) or a Micro Processing Unit (MPU).

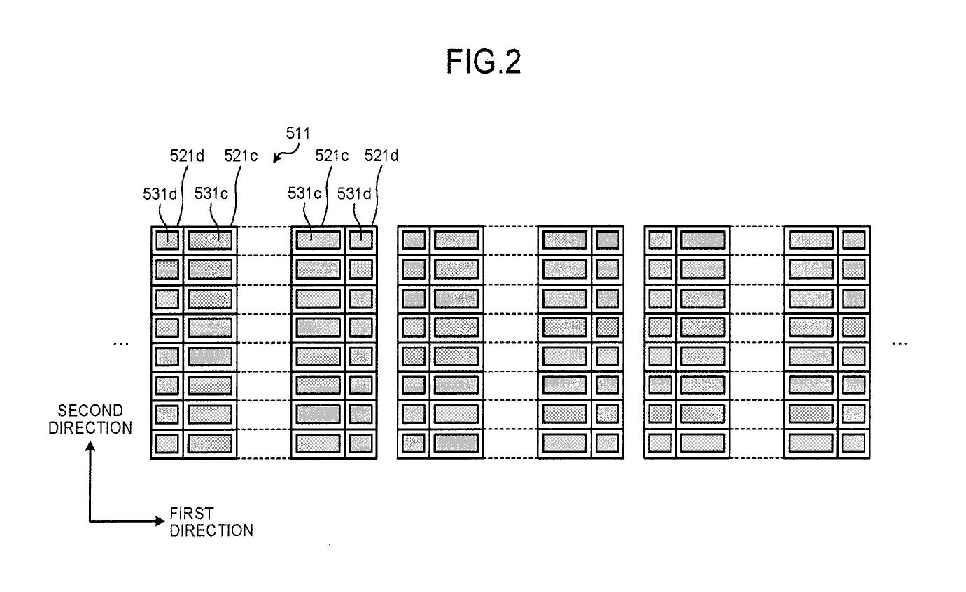

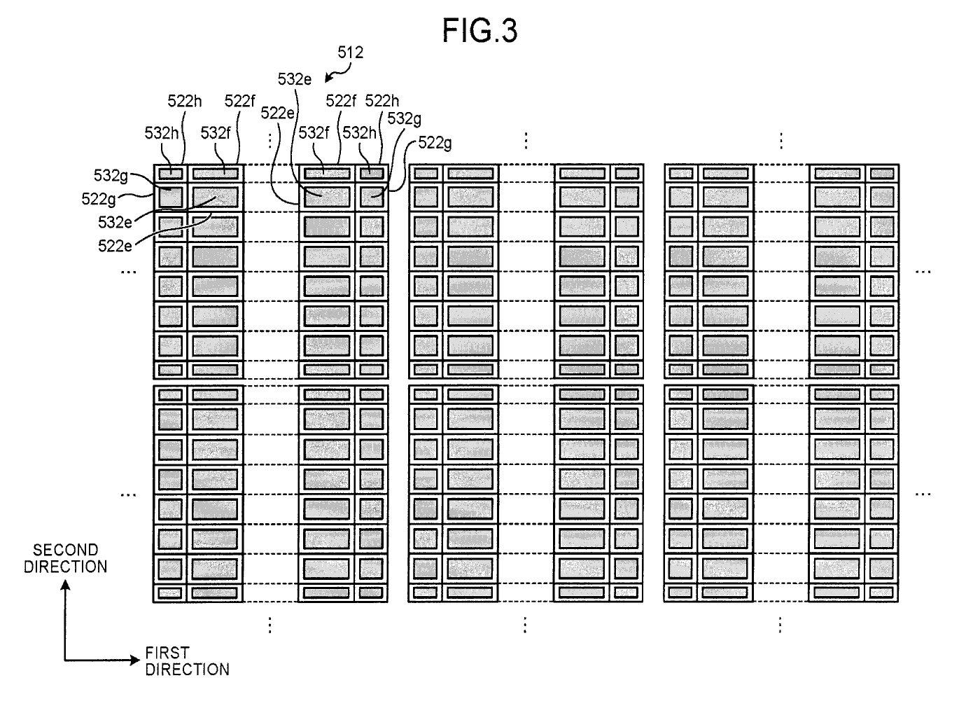

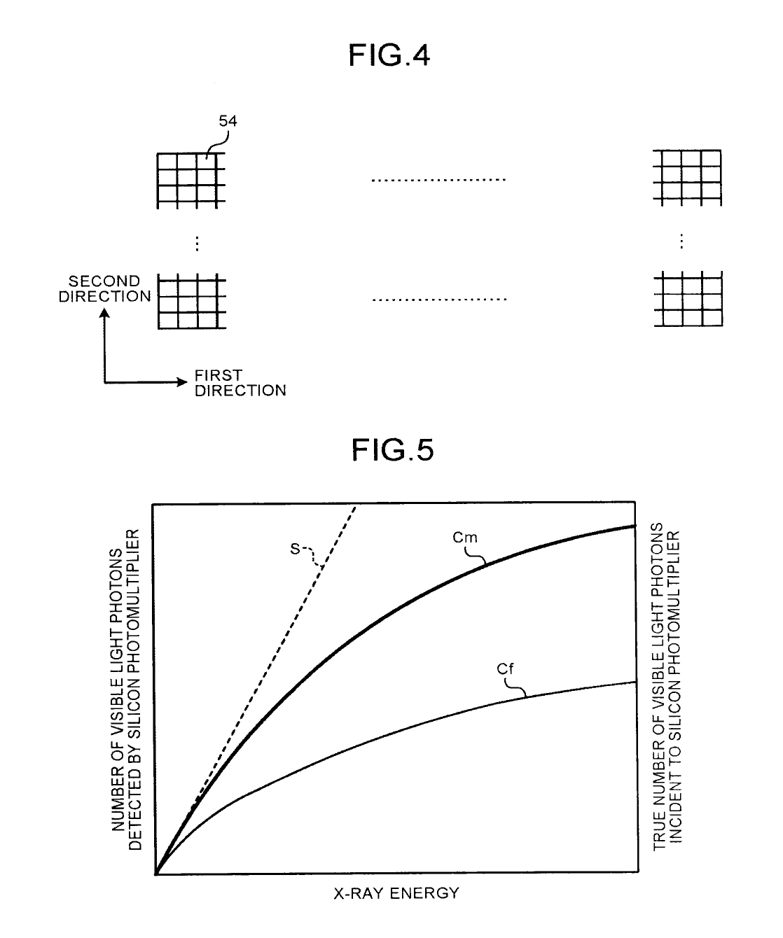

Next, a detector included in conventional medical image diagnostic apparatuses will be explained, with reference to FIGS. 2 to 5. FIG. 2 is a drawing of an example of a configuration and a positional arrangement of SiPM arrays 511 included in a conventional medical image diagnostic apparatus. FIG. 3 is a drawing of another example of a configuration and a positional arrangement of SiPM arrays 512 included in a conventional medical image diagnostic apparatus. FIG. 4 is a diagram of an example of a positional arrangement of cells 54. FIG. 5 is a chart illustrating a relationship between the number of visible light photons detected by a SiPM and X-ray energy and a relationship between the true number of visible light photons that are incident to the SiPM and X-ray energy.

As illustrated in FIG. 2, in the detector included in the conventional medical image diagnostic apparatus, for example, the SiPM arrays 511 are arranged at constant intervals in a first direction. In this situation, the first direction is a channel direction, for example. The channel direction is the circumferential direction of the rotating frame 7.

The SiPM arrays 511 include a plurality of SiPMs 521c and a plurality of SiPMs 521d. As being viewed in the direction perpendicular to the detecting surface of the detector, each of the SiPMs 521c and 521d is in the shape of a rectangle of which one set of opposite sides extend parallel to the first direction, whereas the other set of opposite sides extend parallel to a second direction.

The sides of each of the SiPMs 521d extending parallel to the first direction are shorter than the sides of each of the SiPMs 521c extending parallel to the first direction. This arrangement is made in order to meet both of the following two requirements: One of the requirements is that substrates of the SiPM arrays 511 need to be positioned next to one another at constant intervals in the first direction, for the purpose of preventing the substrates of the SiPM arrays 511 from being damaged mechanically or electrically. The other requirement is that the distances between the centers of any two of the SiPMs positioned adjacent to each other are required to be constant in the first and the second directions, for the purpose of enhancing image quality.

The SiPMs 521d are arranged on two ends, in terms of the first direction, of each of the SiPM arrays 511 and are arranged in the second direction. In this situation, the second direction is, for example, the body axis direction of the subject P. The SiPMs 521c are arranged in a matrix formation in the area interposed between the SiPMs 521d. The SiPMs 521c and the SiPMs 521d correspond to the pixels in each of the views of the projection data described above.

Each of the SiPMs 521c has an effective area 531c. Each of the SiPMs 521d has an effective area 531d. In each of the effective areas 531c and 531d, cells 54 (explained later) are disposed. As being viewed in the direction perpendicular to the detecting surface of the detector, each of the effective areas 531c and 531d is in the shape of a rectangle of which one set of opposite sides extend parallel to the first direction, whereas the other set of opposite sides extend parallel to the second direction.

Further, in the area other than the effective area 531c in each of the SiPMs 521c and the area other than the effective area 531d in each of the SiPMs 521d, wirings connected to the cells 54 and the like are disposed. The wirings connected to the cells 54 are aggregated into one bundle for each of the SiPMs 521c and each of the SiPMs 521d, so as to be connected to the data acquiring unit 6.

Each of the effective areas 531c and 531d is designed so as to have as large an area as possible, so that it is possible to dispose a large number of cells 54 (explained later) therein. Further, as mentioned above, the sides of each of the SiPMs 521d extending parallel to the first direction are shorter than the sides of each of the SiPMs 521c extending parallel to the first direction. Accordingly, the sides of each of the effective areas 531d extending parallel to the first direction are shorter than the sides of each of the effective areas 531c extending parallel to the first direction.

Alternatively, as illustrated in FIG. 3, in a detector included in a conventional medical image diagnostic apparatus, for example, SiPM arrays 512 are arranged at constant intervals in the first direction and in the second direction intersecting the first direction.

Each of the SiPM arrays 512 includes a plurality of SiPMs 522e, a plurality of SiPMs 522f, a plurality of SiPMs 522g, and four SiPMs 522h. As being viewed in the direction perpendicular to the detecting surface of the detector, each of the SiPMs 522e, the SiPMs 522f, the SiPMs 522g, and the SiPMs 522h is in the shape of a rectangle of which one set of opposite sides extend parallel to the first direction, whereas the other set of opposite sides extend parallel to the second direction.

The sides of each of the SiPMs 522f extending parallel to the second direction are shorter than the sides of each of the SiPMs 522e extending parallel to the second direction. The sides of each of the SiPMs 522g extending parallel to the first direction are shorter than the sides of each of the SiPMs 522e extending parallel to the first direction. The sides of each of the SiPMs 522h extending parallel to the first direction are shorter than the sides of each of the SiPMs 522e extending parallel to the first direction and are equal in length to the sides of each of the SiPMs 522g extending parallel to the first direction. The sides of each of the SiPMs 522h extending parallel to the second direction are shorter than the sides of each of the SiPMs 522e extending parallel to the second direction and are equal in length to the sides of each of the SiPMs 522f extending parallel to the second direction. These arrangements are made in order to meet both of the following two requirements: One of the requirements is that substrates of the SiPM arrays 512 need to be positioned next to one another at constant intervals in the first and the second directions, for the purpose of preventing the substrates of the SiPM arrays 512 from being damaged mechanically or electrically. The other requirement is that the distances between the centers of any two of the SiPMs positioned adjacent to each other are required to be constant in the first and the second directions, for the purpose of enhancing image quality.

The SiPMs 522h are arranged in the four corners of each of the SiPM arrays 512. The SiPMs 522f are arranged on two ends, in terms of the second direction, of each of the SiPM arrays 512 and are arranged in the first direction. The SiPMs 522g are arranged on two ends, in terms of the first direction, of each of the SiPM arrays 512 and are arranged in the second direction. The SiPMs 522e are arranged in a matrix formation in the area surrounded by the SiPMs 522f, the SiPMs 522g, and the SiPMs 522h. The SiPMs 522e, the SiPMs 522f, the SiPMs 522g, and the SiPMs 522h correspond to the pixels in each of the views of the projection data described above.

Each of the SiPMs 522e has an effective area 532e. Each of the SiPMs 522f has an effective area 532f. Each of the SiPMs 522g has an effective area 532g. Each of the SiPMs 522h has an effective area 532h. In each of the effective areas 532e, 532f, 532g, and 532h, the cells 54 (explained later) are disposed. As being viewed in the direction perpendicular to the detecting surface of the detector, each of the effective areas 532e, 532f, 532g, and 532h is in the shape of a rectangle of which one set of opposite sides extend parallel to the first direction, whereas the other set of opposite sides extend parallel to the second direction.

Further, in the area other than the effective area 532e in each of the SiPMs 522e, the area other than the effective area 532f in each of the SiPMs 522f, the area other than the effective area 532g in each of the SiPMs 522g, and the area other than the effective area 532h in each of the SiPMs 522h, wirings connected to the cells 54 and the like are disposed. The wirings connected to the cells 54 are aggregated into one bundle for each of the SiPMs 522e, 522f, 522g, and 522h, so as to be connected to the data acquiring unit 6.

Each of the effective areas 532e, 532f, 532g, and 532h is designed so as to have as large an area as possible, so that it is possible to dispose a large number of cells 54 (explained later) therein. Further, the lengths of the sides of each of the SiPMs 522e, 522f, 522g, and 522h have the relationship described above. Accordingly, the lengths of the sides of the effective areas 532e, 532f, 532g, and 532h have the following relationships: The sides of each of the effective areas 532f extending parallel to the second direction are shorter than the sides of each of the effective areas 532e extending parallel to the second direction. The sides of each of the effective areas 532g extending parallel to the first direction are shorter than the sides of each of the effective areas 532e extending parallel to the first direction. The sides of each of the effective areas 532h extending parallel to the first direction are shorter than the sides of each of the effective areas 532e extending parallel to the first direction and are equal in length to the sides of each of the effective areas 532g extending parallel to the first direction. The sides of each of the effective areas 532h extending parallel to the second direction are shorter than the sides of each of the effective areas 532e extending parallel to the second direction and are equal in length to the sides of each of the effective areas 532f extending parallel to the second direction.

As illustrated in FIG. 4, in the effective areas 531c and 531d, or in the effective areas 532e, 532f, 532g, and 532h, the cells 54 are arranged in the first direction and in the second direction intersecting the first direction. Further, the numbers of cells 54 per unit area are equal between the effective areas 531c and the effective areas 531d, or among the effective areas 532e, 532f, 532g, and 532h.

For this reason, in each of the SiPM arrays 511, the number of cells 54 present in each of the SiPMs 521c is different from the number of cells 54 present in each of the SiPMs 521d. Further, in each of the SiPM arrays 512, the number of cells 54 present in each of the SiPMs 522e, the number of cells 54 present in each of the SiPMs 522f, the number of cells 54 present in each of the SiPMs 522g, and the number of cells 54 present in each of the SiPMs 522h are different from one another. When the number of cells 54 is different, the image quality of the projection data and the image quality of the reconstructed image generated by reconstructing the projection data are degraded for the reasons stated below.

The horizontal axis of the chart in FIG. 5 expresses X-ray energy. The vertical axis on the left side of the chart in FIG. 5 expresses the number of visible light photons detected by a SiPM. The vertical axis on the right side of the chart in FIG. 5 expresses the true number of visible light photons that are incident to the SiPM. In this situation, the number of visible light photons released by a scintillator is calculated by dividing the energy of the incident X-rays by a conversion factor of the scintillator. In other words, the energy of the X-rays incident to the scintillator is proportional to the number of visible light photons released by the scintillator. For this reason, it is possible to consider that the horizontal axis of FIG. 5 expresses the number of visible light photons that have become incident to the SiPM.

Ideally, as illustrated with a straight line S in FIG. 5, the true number of visible light photons incident to the SiPM should exhibit a linear behavior with respect to the X-ray energy, i.e., the number of visible light photons generated by the scintillator. However, because the number of cells contained in the SiPM is finite, when the number of visible light photons increases, there is a higher possibility that two or more visible light photons become incident to one cell at the same time. Each of the cells, however, outputs the predetermined electrical signal regardless of whether one visible light photon has become incident thereto or two or more visible light photons have become incident thereto. Thus, some of the visible light photons fail to be counted. Consequently, actuality, as indicated with curves Cm and Cf in FIG. 5, the number of visible light photons detected by the SiPM exhibits a non-linear behavior with respect to the X-ray energy, i.e., the number of visible light photons generated by the scintillator. This is a phenomenon called “pileup”.

Further, the non-linear behavior of the number of visible light photons detected by the SiPM with respect to the X-ray energy is dependent on the number of cells contained in the effective area. The curve Cm in FIG. 5 is a curve indicating the relationship between the X-ray energy and the number of visible light photons detected by the SiPM observed when a larger number of cells are contained in the effective area. The curve Cf in FIG. 5 is a curve indicating the relationship between the X-ray energy and the number of visible light photons detected by the SiPM observed when a smaller number of cells are contained in the effective area. As the curves Cm and Cf are compared with each other, it is understood that, when the smaller number of cells are contained in the effective area, the number of visible light photons detected by the SiPM with respect to the X-ray energy exhibits a non-linear behavior, starting at a lower level of X-ray energy. This is because the smaller the number of cells contained in the effective area is, the more easily the pileup phenomenon occurs.

Consequently, when the conventional medical image diagnostic apparatus including the SiPM arrays containing SiPMs that have the mutually-different effective area sizes is used, the image quality may be degraded by the difference in the behaviors of the number of visible light photons detected by the SiPM with respect to the X-ray energy.

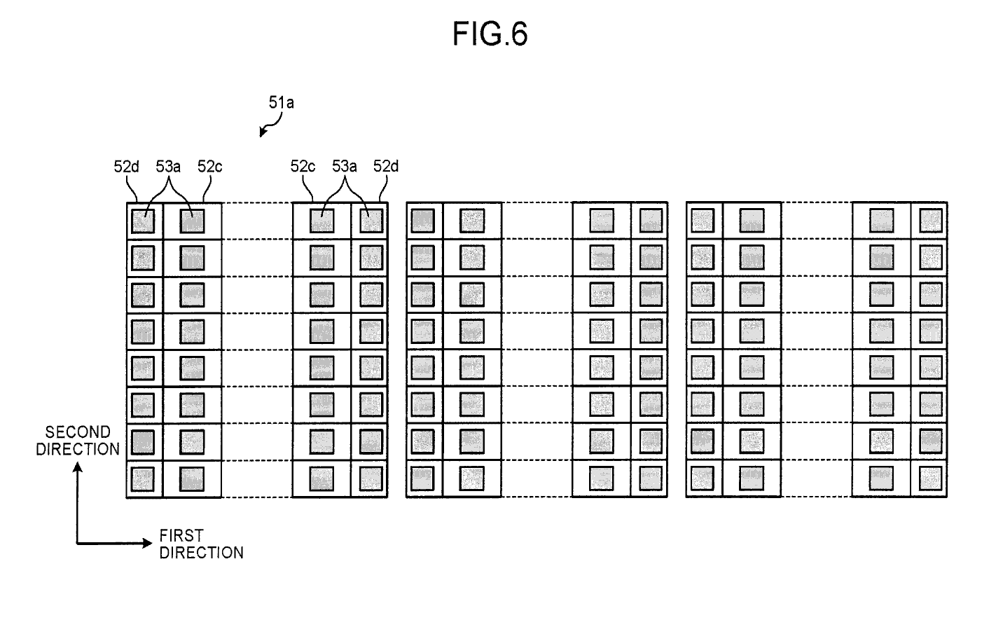

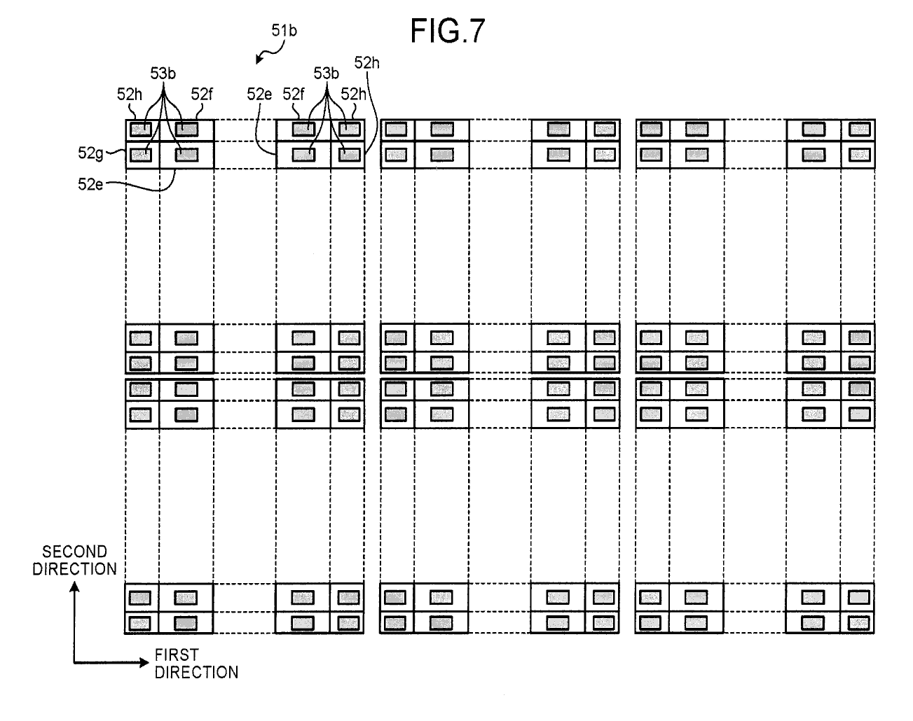

Next, the detector 5 included in the medical image diagnostic apparatus 1 according to the present embodiment will be explained, with reference to FIGS. 6 and 7. FIG. 6 is a drawing of an example of a configuration and a positional arrangement of SiPM arrays 51a included in the medical image diagnostic apparatus 1 according to the present embodiment. FIG. 7 is a drawing of an example of a configuration and a positional arrangement of SiPM arrays 51b included in the medical image diagnostic apparatus 1 according to the present embodiment.

As illustrated in FIG. 6, in the detector 5 included in the medical image diagnostic apparatus 1 according to the present embodiment, for example, the SiPM arrays 51a are arranged at constant intervals in the first direction. In this situation, the first direction is the channel direction, for example. The channel direction is the circumferential direction of the rotating frame 7.

The SiPM arrays 51a include a plurality of SiPMs 52c and a plurality of SiPMs 52d. As being viewed in the direction perpendicular to the detecting surface of the detector 5, each of the SiPMs 52c and 52d is in the shape of a rectangle of which one set of opposite sides extend parallel to the first direction, whereas the other set of opposite sides extend parallel to the second direction.

The sides of each of the SiPMs 52d extending parallel to the first direction are shorter than the sides of each of the SiPMs 52c extending parallel to the first direction. This arrangement is made for the same reason as with the arrangement of the SiPM arrays 511 in the first direction illustrated in FIG. 2.

The SiPMs 52d are arranged on two ends, in terms of the first direction, of each of the SiPM arrays 51a and are arranged in the second direction. In this situation, the second direction is, for example, the body axis direction of the subject P. The SiPMs 52c are arranged in a matrix formation in the area interposed between the SiPMs 52d. The SiPMs 52c and the SiPMs 52d correspond to the pixels in each of the views of the projection data described above.

Each of the SiPMs 52c and the SiPMs 52d has an effective area 53a. In each of the effective areas 53a, the cells 54 described above are disposed. As being viewed in the direction perpendicular to the detecting surface of the detector 5, each of the effective areas 53a is in the shape of a rectangle of which one set of opposite sides extend parallel to the first direction, whereas the other set of opposite sides extend parallel to the second direction. The number of cells 54 contained in each of the SiPMs 52c is equal to the number of cells 54 contained in each of the SiPMs 52d.

Further, in the area other than the effective area 53a in each of the SiPMs 52c and the SiPMs 52d, wirings connected to the cells 54 and the like are disposed. The wirings connected to the cells 54 are aggregated into one bundle for each of the SiPMs 52c and each of the SiPMs 52d, so as to be connected to the data acquiring unit 6. Alternatively, as illustrated in FIG. 7, in the detector 5 included in the medical image diagnostic apparatus 1 according to the present embodiment, for example, the SiPM arrays 51b are arranged at constant intervals in the first direction and in the second direction intersecting the first direction.

Each of the SiPM arrays 51b includes a plurality of SiPMs 52e, a plurality of SiPMs 52f, a plurality of SiPMs 52g, and four SiPMs 52h. As being viewed in the direction perpendicular to the detecting surface of the detector 5, each of the SiPMs 52e, the SiPMs 52f, the SiPMs 52g, and the SiPMs 52h is in the shape of a rectangle of which one set of opposite sides extend parallel to the first direction, whereas the other set of opposite sides extend parallel to the second direction.

The sides of each of the SiPMs 52f extending parallel to the second direction are shorter than the sides of each of the SiPMs 52e extending parallel to the second direction. The sides of each of the SiPMs 52g extending parallel to the first direction are shorter than the sides of each of the SiPMs 52e extending parallel to the first direction. The sides of each of the SiPMs 52h extending parallel to the first direction are shorter than the sides of each of the SiPMs 52e extending parallel to the first direction and are equal in length to the sides of each of the SiPMs 52g extending parallel to the first direction. The sides of each of the SiPMs 52h extending parallel to the second direction are shorter than the sides of each of the SiPMs 52e extending parallel to the second direction and are equal in length to the sides of each of the SiPMs 52f extending parallel to the second direction. These arrangements are made for the same reason as with the arrangement of the SiPM arrays 512 in the first and the second directions illustrated in FIG. 3.

The SiPMs 52h are arranged in the four corners of each of the SiPM arrays 51h. The SiPMs 52f are arranged on two ends, in terms of the second direction, of each of the SiPM arrays 51b and are arranged in the first direction. The SiPMs 52g are arranged on two ends, in terms of the first direction, of each of the SiPM arrays 51b and are arranged in the second direction. The SiPMs 52e are arranged in a matrix formation in the area surrounded by the SiPMs 52f, the SiPMs 52g, and the SiPMs 52h. The SiPMs 52e, the SiPMs 52f, the SiPMs 52g, and the SiPMs 52h correspond to the pixels in each of the views of the projection data described above.

Each of the SiPMs 52e, 52f, 52g, and 52h has an effective area 53b. In each of the effective areas 53b, the cells 54 described above are disposed. As being viewed in the direction perpendicular to the detecting surface of the detector 5, each of the effective areas 53b is in the shape of a rectangle of which one set of opposite sides extend parallel to the first direction, whereas the other set of opposite sides extend parallel to the second direction. The number of cells 54 contained in each of the SiPMs 52e, the number of cells 54 contained in each of the SiPMs 52f, the number of cells 54 contained in each of the SiPMs 52g, and the number of cells 54 contained in each of the SiPMs 52h are equal to one another.

Further, in the area other than the effective area 53b in each of the SiPMs 52e, 52f, 52g, and 52h, wirings connected to the cells 54 and the like are disposed. The wirings connected to the cells 54 are aggregated into one bundle for each of the SiPMs 52e, 52f, 52g, and 52h, so as to be connected to the data acquiring unit 6. Further, the visible light generated as a result of the X-rays becoming incident to the scintillators is not necessarily released from the scintillators in a spatially uniform manner. For this reason, in the detector 5, it is desirable that effective areas 53b contain an equal number of cells per unit area, while allowing for a margin of dimensional errors that may occur during the manufacture. Alternatively, in the detector 5, it is desirable that effective areas 53b contain an equal number of cells per unit area.

Further, in the detector 5, it is desirable that the shapes of the effective areas are the same as one another, while allowing for a margin of dimensional errors that may occur during the manufacture. Alternatively, in the detector 5, it is desirable that the shapes of the effective areas are the same as one another.

If the detector 5 satisfies at least one of these configurations, even if the visible light is not released from the scintillators in a spatially uniform manner, it is possible to inhibit the occurrence of the pileup phenomenon that may be caused when visible light photons enter only some of the cells in a concentrated manner. Consequently, the medical image diagnostic apparatus 1 including the detector 5 is able to prevent the image quality from being degraded.

Further, in the detector 5, it is desirable that the effective areas are positioned at regular intervals in the first direction and in the second direction intersecting the first direction, while allowing for a margin of dimensional errors that may occur during the manufacture. Alternatively, in the detector 5, it is desirable that the effective areas are positioned at regular intervals in the first direction and in the second direction intersecting the first direction. In this situation, as mentioned above, the first direction is the channel direction, whereas the second direction is the body axis direction of the subject, for example. Further, in the detector 5, it is desirable that the distance between any two effective areas that are positioned adjacent to each other while respectively belonging to two adjacently-positioned SiPM arrays is equal to the distance between any two effective areas positioned adjacent to each other while belonging to mutually the same SiPM arrays. For example, when the SiPM arrays 51a are arranged as illustrated in FIG. 6, the distance between the effective area 53a positioned at the right end of the SiPM array 51a positioned at the left end and the effective area 53a positioned at the left end of the SiPM array 51a positioned in the middle is equal to the distance between the effective areas 53a positioned adjacent to each other in the first direction while belonging to mutually the same SiPM array 51a. The same also applies to the situation where the SiPM arrays 51b are arranged as illustrated in FIG. 7. Further, when the SiPM arrays 51b are arranged as illustrated in FIG. 7, the distance between the effective area 53b positioned at the lower end of the SiPM array 51b positioned in the upper middle section and the effective area 53b positioned at the upper end of the SiPM array 51b positioned in the lower middle section is equal to the distance between the effective areas 53b positioned adjacent to each other in the second direction while belonging to mutually the same SiPM array 51b. When at least one of these configurations is satisfied, because the positional arrangement of the effective areas in the detector 5 becomes close to a uniform positional arrangement, the medical image diagnostic apparatus 1 including the detector 5 is able to prevent the image quality from being degraded.

As illustrated in FIG. 6, each of the effective areas 53a contained in the SiPMs 52d does not necessarily have to be positioned at the center in the first direction. For example, as illustrated in FIG. 6, each of the effective areas 53a contained in the SiPMs 52d may be arranged so as to be positioned close to the space between the two SiPM arrays 51a positioned adjacent to each other. By adjusting the positions of the effective areas contained in the SiPMs 52d appropriately, it is possible to arrange the effective areas to be positioned at regular intervals in the first direction, even when it is not possible to reserve sufficient spaces between the SiPM arrays 51a.

Further, as illustrated in FIG. 7, each of the effective areas 53b contained in the SiPMs 52f does not necessarily have to be positioned at the center in the second direction. Similarly, each of the effective areas 53b contained in the SiPMs 52g does not necessarily have to be positioned at the center in the first direction. Also, each of the effective areas 53b contained in the SiPMs 52h does not necessarily have to be positioned at the center in one or both of the first and the second directions. In other words, each of the effective areas 53b contained in the SiPMs 52f, 52g, and 52h may be arranged so as to be positioned close to the space between the two SiPM arrays 51b positioned adjacent to each other. By adjusting the positions of the effective areas contained in the SiPMs 52f, 52g, and 52h appropriately, it is possible to arrange the effective areas to be positioned at regular intervals in the first direction and in the second direction intersecting the first direction, even when it is not possible to reserve sufficient spaces between the SiPM arrays 51b.

According to the embodiment described above, in the detector 5, the number of cells 54 contained in each of the SiPMs 52c is equal to the number of cells 54 contained in each of the SiPMs 52d. Further, according to the embodiment described above, in the detector 5, the number of cells 54 contained in each of the SiPMs 52e, the number of cells 54 contained in each of the SiPMs 52f, the number of cells 54 contained in each of the SiPMs 52g, and the number of cells 54 contained in each of the SiPMs 52h are equal to one another.

Consequently, the behavior of the number of visible light photons detected by the SiPMs 52c with respect to the X-ray energy is equal to the behavior of the number of visible light photons detected by the SiPMs 52d with respect to the X-ray energy. Further, the behavior of the number of visible light photons detected by the SiPMs 52e with respect to the X-ray energy, the behavior of the number of visible light photons detected by the SiPMs 52f with respect to the X-ray energy, the behavior of the number of visible light photons detected by the SiPMs 52g with respect to the X-ray energy, the behavior of the number of visible light photons detected by the SiPMs 52h with respect to the X-ray energy are equal to one another. Consequently, the medical image diagnostic apparatus 1 including the detector 5 is able to prevent the image quality from being degraded by the difference in the behaviors of the number of visible light photons detected by the SiPMs with respect to the X-ray energy.

In the detector 5, the effective areas do not necessarily have to be arranged to be positioned at regular intervals in the first direction and the second direction intersecting the first direction. Further, in the detector 5, the shapes of the effective areas may be different from one another. Further, in the detector 5, the area sizes of the effective areas may be different from one another. In other words, as long as at least the numbers of cells contained in the SiPMs are equal, the medical image diagnostic apparatus 1 is able to achieve the effect described above.

Further, the detector 5 described above may be employed not only in a photon-counting-type X-ray CT apparatus but also in a nuclear medical imaging apparatus such as a PET apparatus or a SPECT apparatus, or an X-ray diagnostic apparatus that includes a photon-counting-type detector. In the detector 5 described above, because the behaviors of the number of visible light photons detected by the SiPMs with respect to the energy of the radiation are equal among all the energy ranges, the detector 5 is particularly effective in use in a medical image diagnostic apparatus that uses radiation in a large energy range.

The constituent elements described above are based on functional concepts. Thus, it is not necessary to physically configure the elements as indicated in the FIG. 1. In other words, the specific mode of distribution and integration of the constituent elements is not limited to the one illustrated in FIG. 1. It is acceptable to functionally or physically distribute or integrate all or a part of the constituent elements in any arbitrary units, depending on various loads and the status of use. Further, all or an arbitrary part of the processing functions performed by the constituent elements may be realized by a CPU and a computer program executed by the CPU. Alternatively, all or an arbitrary part of the processing functions performed by the constituent elements may be realized as hardware using wired logic.

### Modified Example

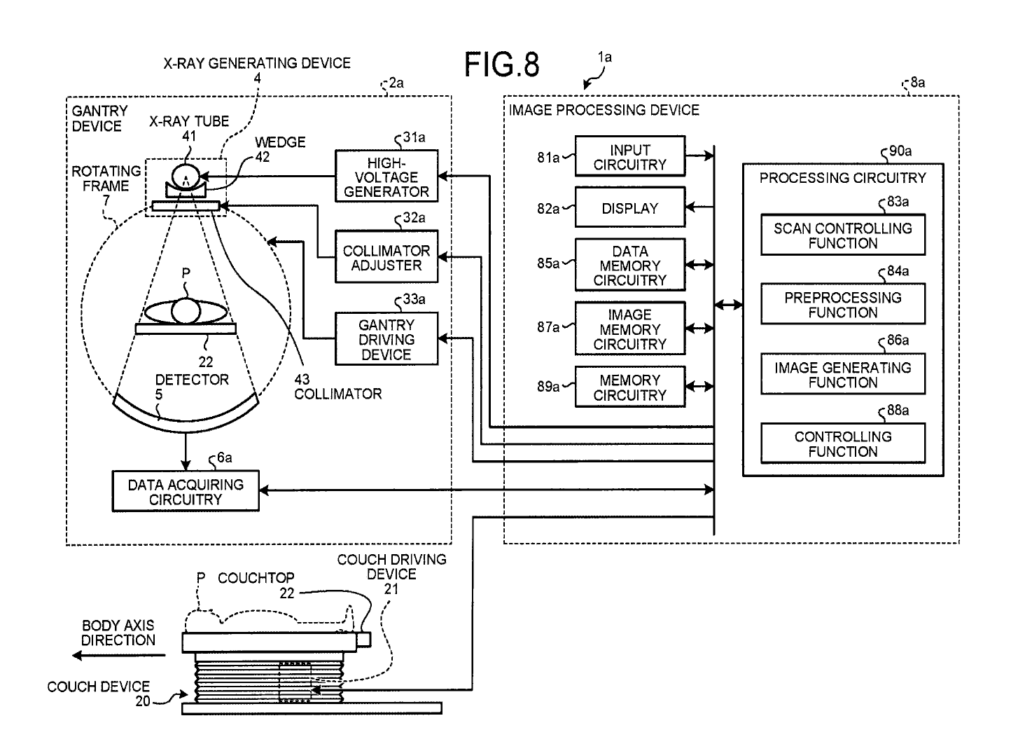

A modified example of the embodiment described above will be explained, with reference to FIG. 8. FIG. 8 is a diagram of an exemplary configuration of a medical image diagnostic apparatus 1a according to a modified example. The medical image diagnostic apparatus 1a is a photon-counting-type X-ray CT apparatus. Some of the elements that are the same as those in the embodiment described above will be referred to by using the same reference characters as those in the embodiment described above. Further, for some of the contents that are duplicates of those in the embodiment described above, detailed explanation will be omitted. As illustrated in FIG. 8, the medical image diagnostic apparatus 1a includes a gantry device 2a, the couch device 20, and an image processing device 8a.

The gantry device 2a acquires projection data by irradiating X-rays with the subject P. The gantry device 2a includes a high-voltage generator 31a, a collimator adjuster 32a, a gantry driving device 33a, the X-ray generating device 4, the detector 5, data acquiring circuitry 6a, and the rotating frame 7.

The high-voltage generator 31a supplies an X-ray tube voltage to the X-ray tube 41. The collimator adjuster 32a adjusts the radiating range of the X-rays radiated by the X-ray generating device 4 and irradiated the subject P, by adjusting the degree of aperture and the position of the collimator 43. The gantry driving device 33a causes the X-ray generating device 4 and the detector 5 to rotate a circular trajectory centered on the subject P, by driving the rotating frame 7 to rotate.

The gantry driving device 33a includes, for example, a motor, an electronic circuit, and a driving mechanism. The motor generates a power for causing the rotating frame 7 to rotate. The electronic circuit controls operations of the motor. The driving mechanism converts the power generated by the motor into a power that causes the rotating frame 7 to rotate. The driving mechanism is realized with a combination of, for example, gears, belts, shafts, bearings, and the like. The rotating frame 7 is configured, in collaboration with the gantry driving device 33a, to cause the X-ray tube 41 and the detector 5 to rotate.

The data acquiring circuitry 6a has the same functions as those of the data acquiring unit 6 described in the embodiment above. The data acquiring circuitry 6a acquires the count data described above. Specifically, the data acquiring circuitry 6a performs the count data acquiring operation by reading and executing a computer program (hereinafter, “program”) stored in memory circuitry 89a (explained later). Further, the data acquiring circuitry 6a is realized by using a processor.

The image processing device 8a includes input circuitry 81a, a display 82a, data memory circuitry 85a, image memory circuitry 87a, processing circuitry 90a, and the memory circuitry 89a.

For example, the input circuitry 81a is realized with a mouse, a keyboard, and/or the like used by the user of the medical image diagnostic apparatus 1a for inputting various types of instructions and various types of settings. The input circuitry 81a outputs the various types of instructions and the various types of settings input by the user to the processing circuitry 90a (explained later) as electrical signals. The input circuitry 81a has the same functions as those of the input unit 81 described in the embodiment above.

On the basis of the electrical signals received from the processing circuitry 90a (explained later), the display 82a displays results of various types of image processing, a Graphical User Interface (GUI) used for receiving the various types of settings from the user via the input circuitry 81a, and the like. For example, the display 82a may be a liquid crystal display or an organic Electroluminescence (EL) display. The display 82a has the same functions as those of the display unit 82 described in the embodiment above.

The data memory circuitry 85a stores therein raw data generated by a preprocessing function 84a (explained later). The data memory circuitry 85a has the same functions as those of the data storage unit 85 described in the embodiment above.

The image memory circuitry 87a stores therein a CT image generated by an image generating function 86a (explained later). The image memory circuitry 87a has the same functions as those of the image storage unit 87 described in the embodiment above.

The memory circuitry 89a has stored therein programs for realizing a scan controlling function 83a, the preprocessing function 84a, the image generating function 86a, and a controlling function 88a. Further, the memory circuitry 89a has stored therein a program used by the data acquiring circuitry 6a to realize the functions of the data acquiring unit 6.

The processing circuitry 90a performs the same processes as those performed by the scan controlling unit 83, by reading and executing a program corresponding to the scan controlling function 83a from the memory circuitry 89a. Further, the processing circuitry 90a performs the same processes as those performed by the preprocessing unit 84, by reading and executing a program corresponding to the preprocessing function 84a from the memory circuitry 89a. Further, the processing circuitry 90a performs the same processes as those performed by the image generating unit 86, by reading and executing a program corresponding to the image generating function 86a from the memory circuitry 89a. Furthermore, the processing circuitry 90a performs the same processes as those performed by the controlling unit 88, by reading and executing a program corresponding to the controlling function 88a from the memory circuitry 89a. The processing circuitry 90a according to the present modified example is an example of the processing circuitry in the claims.

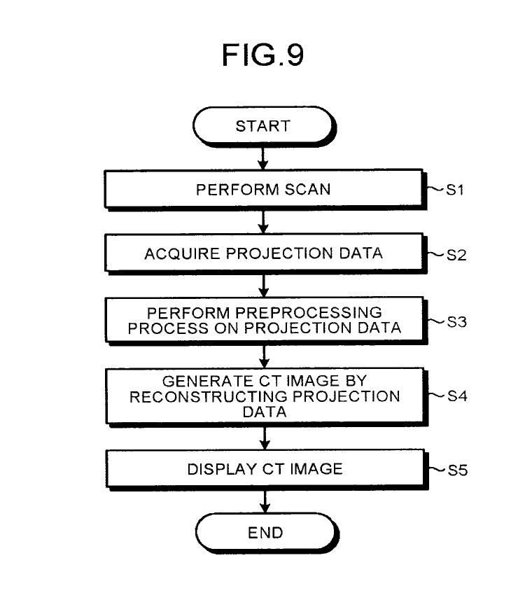

Next, a process performed by the medical image diagnostic apparatus 1a according to the modified example will be explained, with reference to FIG. 9. FIG. 9 is a flowchart of an example of the process performed by the medical image diagnostic apparatus 1a according to the modified example.

Step S1 in FIG. 9 is a step realized by the processing circuitry 90a while reading and executing the program corresponding to the scan controlling function 83a from the memory circuitry 89a. At step S1, according to the scan controlling function 83a executed by the processing circuitry 90a, the gantry device 2a performs a scan.

Step S2 is a step realized by the data acquiring circuitry 6a while reading and executing the data acquiring program from the memory circuitry 89a. At step S2, the data acquiring circuitry 6a acquires the projection data.

Step S3 is a step realized by the processing circuitry 90a while reading and executing the program corresponding to the preprocessing function 84a from the memory circuitry 89a. At step S3, the processing circuitry 90a performs preprocessing process on the projection data.

Step S4 is a step realized by the processing circuitry 90a while reading and executing the program corresponding to the image generating function 86a from the memory circuitry 89a. At step S4, the processing circuitry 90a generates a CT image by reconstructing the projection data.

Step S5 is a step realized by the processing circuitry 90a while reading and executing the program corresponding to the controlling function 88a from the memory circuitry 89a. At step S5, the display 82a displays the CT image according to the controlling function 88a executed by the processing circuitry 90a.

The processor described above may be, for example, a Central Processing Unit (CPU), a Graphics Processing Unit (GPU), an Application Specific Integrated Circuit (ASIC), a Programmable Logic Device (PLD), or a Field Programmable Gate Array (FPGA). Further, the Programmable Logic Device (PLD) may be, for example, a Simple Programmable Logic Device (SPLD) or a Complex Programmable Logic Device (CPLD).

The processor realizes the functions thereof by reading and executing the programs stored in the memory circuitry 89a. In the modified example described above, the single piece of processing circuitry (the processing circuitry 90a) realizes the scan controlling function 83a, the preprocessing function 84a, the image generating function 86a, and the controlling function 88a. However, in the modified example, the processing circuitry 90a may be configured by combining a plurality of independent processors together. Alternatively, in the modification described above, the scan controlling function 83a, the preprocessing function 84a, the image generating function 86a, and the controlling function 88a may each be realized with independent processing circuitry. Alternatively, in the modified example described above, it is acceptable to arbitrarily integrate together any of the processing circuitry elements realizing the scan controlling function 83a, the preprocessing function 84a, the image generating function 86a, and the controlling function 88a.

According to at least one aspect of the embodiments described herein, it is possible to prevent the image quality from being degraded.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising:
detecting elements each of which contains, in an effective area thereof, a plurality of cells each configured to output an electrical signal when at least one photon has become incident thereto; and
processing circuitry configured to generate an image based on a signal obtained by adding together the electrical signals output by the plurality of cells, wherein
the medical image diagnostic apparatus includes a plurality of arrays in each of which two or more of the detecting elements containing an equal number of cells in the effective areas thereof are arranged, and
the plurality of arrays are arranged in such a manner that distances between centers of the effective areas are constant.

2. The medical image diagnostic apparatus according to claim 1, wherein, in a detector that includes the plurality of arrays, the effective areas contain an equal number of cells per unit area.

3. The medical image diagnostic apparatus according to claim 1, wherein, in a detector that includes the plurality of arrays, shapes of the effective areas are same as one another.

4. The medical image diagnostic apparatus according to claim 2, wherein, in the detector that includes the plurality of arrays, shapes of the effective areas are same as one another.

5. The medical image diagnostic apparatus according to claim 1, wherein, in a detector that includes the plurality of arrays, the effective areas are arranged to be positioned at regular intervals in a first direction that is a channel direction and in a second direction that is a body axis direction of a subject and that intersects the first direction.

6. The medical image diagnostic apparatus according to claim 2, wherein, in the detector that includes the plurality of arrays, the effective areas are arranged to be positioned at regular intervals in a first direction that is a channel direction and in a second direction that is a body axis direction of a subject and that intersects the first direction.

7. The medical image diagnostic apparatus according to claim 3, wherein, in the detector that includes the plurality of arrays, the effective areas are arranged to be positioned at regular intervals in a first direction that is a channel direction and in a second direction that is a body axis direction of a subject and that intersects the first direction.

8. The medical image diagnostic apparatus according to claim 4, wherein, in the detector that includes the plurality of arrays, the effective areas are arranged to be positioned at regular intervals in a first direction that is a channel direction and in a second direction that is a body axis direction of a subject and that intersects the first direction.

9. An X-ray CT apparatus comprising:
an X-ray tube configured to generate X-rays to be irradiated a subject;
a detector that includes detecting elements each of which contains, in an effective area thereof, a plurality of cells each configured to output an electrical signal when at least one photon has become incident thereto, the detector including a plurality of arrays in each of which two or more of the detecting elements containing an equal number of cells in the effective areas thereof are arranged, while the plurality of arrays are arranged in such a manner that distances between centers of the effective areas are constant;
a rotating frame configured to cause the X-ray tube and the detector to rotate; and
processing circuitry configured to generate a reconstructed image based on the signals obtained from the detector.

10. A detector comprising detecting elements each of which contains, in an effective area thereof, a plurality of cells each configured to output an electrical signal when at least one photon has become incident thereto, wherein
the detector includes a plurality of arrays in each of which two or more of the detecting elements containing an equal number of cells in the effective areas thereof are arranged, while the plurality of arrays are arranged in such a manner that distances between centers of the effective areas are constant.
